(19) United States
(12) Patent Application Publication
(10) Pub. No.: US 2024/0268417 A1
(43) Pub. Date: Aug. 15, 2024

(54) INCLUSION OF SEAWEED IN RUMINANT FEEDSTOCK FOR REDUCING METHANE PRODUCTION AND INCREASING CONSUMER PRODUCT QUALITY

(71) Applicant: CASCADIA SEAWEED CORP., Sidney (CA)

(72) Inventor: Spencer SERIN, Courtenay, BC (CA)

(73) Assignee: CASCADIA SEAWEED CORP., Sidney, BC (CA)

(21) Appl. No.: 18/559,538

(22) PCT Filed: May 16, 2022
(86) PCT No.: PCT/CA2022/050771

§ 371 Date: Nov. 7, 2023

(30) Foreign Application Priority Data
May 17, 2021 (CA) 3118811

(51) Int. Cl. A23K 10/30; A23K 30/12; A23K 50/10

(52) U.S. Cl. CPC A23K 10/30; A23K 30/12; A23K 50/10

(57) ABSTRACT

A feed additive is provided for adding to a ruminant feedstock, the feed additive comprising a dried, powdered mixture of at least two macroalgae selected from the group consisting of ocean farm-grown Egregia menziesii, Neoagarum fimbriatum, Saccharina latissimi, Alaria marginate, Macrocystis pyrifera, Hedophyllum sessile, Callophyllis species, Opuntiella californica, Sarcodiotheca gaudichaudii, and Mazzaella splendins. A method of manufacturing the feed additive is also provided as is use of the feed additive.

### FIELD

The present technology is directed to developing ruminant feeds that include seaweed, both to reduce methane production in the ruminants and to improve meat and milk quality. More specifically it is directed to seaweeds that are native to the northern Pacific Ocean proximate to or at the west coast of Canada and are cultivated.

### BACKGROUND

As disclosed in Seaweed and Seaweed Bioactives for Mitigation of Enteric Methane: Challenges and Opportunities (D. Wade Abbott et al, Animals (Basel). 2020 Dec; 10(12): 2432. Published online 2020 Dec 18. Doi: 10.3390/ani10122432), Alaria esculenta, a brown seaweed, decreased methane production in an in vitro system. The decrease was linearly related to the content of the seaweed.

However, in another study, (Molina-Alcaide E., Carro M. D., Roleda M. Y., Weisbjerg M. R., Lind V., Novoa-Garrido M. In vitro ruminal fermentation and methane production of different seaweed species. Anim. Feed Sci. Technol. 2017;228:1-12. Doi: 10.1016/j.anifeedsci.2017.03.012.) Alaria esculenta was found to have no effect on methane production.

As disclosed in The Potential Role of Seaweeds in the Natural Manipulation of Rumen Fermentation and Methane Production (Margarida R. G. Maia et al www.nature.com/scientificreports), Saccharina latisma either had no effect on methane production when added to meadow hay, or increased methane production when added to corn silage.

As disclosed in https://www.linkedin.com/pulse/giant-kelp-bovine-beano-phil-cruver/California's Giant kelp (Macrocystis pyrifera) has special powers to reduce methane as both an environmentally responsible effort and a nutritional way to improve efficiency of feed utilization in livestock. In the laboratory, the digestive processes of cattle with 2% of the feed as seaweed, the methane production was reduced to undetectable levels greater than 99%.

U.S. Pat. Nos. 10,881,697 and 9,980,995 and United States Patent Application Publication No. 20210077555 disclose a method of reducing total gas production and/or methane production in a ruminant animal. The red algae, Asparagopsis armata and Asparagopsis taxiformis were shown to reduce methane production in steers. In an in vitro system, the Asparagopsis species, Dictyota bartayresii and Cladophora patentiramea also had the most pronounced effect on reducing in vitro. Asparagopsis armata and Asparagopsis taxiformis are red macroalgae, Dictyota bartayresii is a brown macroalgae and Cladophora patentiramea is a green macroalgae. Asparagopsis species are native to South Australia but and are invasive species in the Northern hemisphere. Dictyota bartayresii is found in the tropical western Indo-Pacific region and the Gulf of Mexico. Cladophora patentiramea grows in the Mediterranean Sea and again is a tropical macroalgae. A barrier to commercialization of Asparagopsis is the mass production of this specific macroalgal biomass at a scale to provide supplementation to livestock as the individual algae are small. Further, at present, none of the Asparagopsis species, Dictyota bartayresii and Cladophora patentiramea are cultivated. Another area requiring characterization is the most appropriate method for processing (dehydration) and feeding to livestock in systems with variable feed quality and content.

In a more recent publication, the following macroalgae were tested at 5% in the feedstock: Asparagopsis armata; Asparagopsis taxiformis Caulerpa taxifolia; Cladophora patentiramea; Cystoseria trinodis; Dictyota bartayresii; Padina australis; Sargassum flavicans; and Ulva ohnoi. The results showed improvements in volatile fatty acids however, there was a minimal effect on gas production and no clear justification for a ranking order were demonstrated. When tested in combination with Asparagopsis, the effects on fermentation were dominated by presence of Asparagopsis at 2% and no further benefits demonstrated. Therefore, Asparagopsis remains the only macroalga inducing near elimination of methane in vitro and benefit of combinations with other macroalgae evaluated in this study was not demonstrated. (Kinley, R. D., Vucko, M. J., Machado, L. and Tomkins, N. W. (2016), In Vitro Evaluation of the Antimethanogenic Potency and Effects on Fermentation of Individual and Combinations of Marine Macroalgae. American Journal of Plant Sciences, 7, 2038-2054.)

In all these studies the effect of growth conditions on the efficacy of the macroalgae to reduce methane production in ruminants is unknown as the macroalgae are not grown commercially and are harvested from wild stock. Further, there does not appear to be a consensus of results for any macroalgae except Asparagopsis taxiformis. Hence, it is not known if other genera of macroalgae can be added to feedstock to reduce methane production in ruminants.

United States Patent Application Publication No. 20030003134 discloses that a seaweed supplement from Ascophylum nodosum is included in diet of mammals and poultry to enhance immune response. In one embodiment, pasture forage is treated with seaweed supplement. In another embodiment, seaweed meal is directly fed to mammals and enhanced immune responses is manifested by increased width response after intradermal injection of phytohemaglutinin. In an independently inventive embodiment, seaweed supplement is administered to pigs exposed to porcine reproductive and respiratory syndrome disease to impart resistance to said disease and improve performance. In stilt another independently inventive embodiment, seaweed supplement is administered to lactating mares prior to weaning to mitigate the stress of weaning.

In another study, Ascophyllum nodosum was fed to steers. Steers were housed in four groups of eight and received Tasco-14™ in the diet, in place of barley, at levels (as fed) of 10 g/kg for 14 days (T1-14), 20 g/kg for 7 days (T2-7), 20 g/kg for 14 days (T2-14), or not at all (i.e., control, CON). The dietary treatments commenced 7 days after E. coli O157:H7 inoculation and fecal shedding patterns were examined over 14 weeks. Water, water-trough interface, feed and fecal pat samples were also collected weekly and cultured for E. coli O157:H7. Detection of the pathogen in fecal samples was less frequent (P<0.05) in T1-14 (99/168) and T2-7 (84/168) versus CON (135/168) and T2-14 (115/168), and the concentrations of E. coli O157:H7 recovered in feces from T1-14 and T2-7 steers were lower (P<0.005) than from CON or T2-14 steers. Rates of decline in shedding of E. coli O157:H7 were similar among treatments, but final numbers of E. coli O157:H7 were lower (P<0.05) in T1-14 and T2-7 as compared to T2-14 and CON. (Effect of feeding sun-dried seaweed (Ascophyllum nodosum) on fecal shedding of Escherichia coli O157:H7 by feedlot cattle and on growth performance of lambs, Animal Feed Science and Technology, Volume 142, Issues 1-2, 15 April 2008, Pages 17-32).

There are numerous prior art documents that disclose feedstocks or feed additives to improve animal performance with regard to growth rate, meat quality or milk quality. For example United States Patent Application Publication No. 20200281225 discloses a feed additive composition consisting essentially of a direct fed microbial comprising one or more bacterial strains in combination with at least one protease and a method for improving the performance of a subject or for improving digestibility of a raw material in a feed, which method comprising administering to a subject a direct fed microbial in combination with a protease.

What is needed is a feedstock additive that improves ruminant performance with regard to one or more of growth rate, meat quality and meat quantity, or milk quality. It would be preferable if it reduces methane production and reduced bacterial count in ruminants. It would be preferable if the feedstock additive included at least one brown or red algae species native to the northeastern Pacific Ocean. It would be also preferable to have a feedstock additive that included at least one brown or red algae species native to the northwestern Atlantic Ocean. As we have learned from fish farming, there are significant negative environmental implications of farming non-native species. It would be further preferable if the additive was derived from macroalgae that can be cultivated in the northeastern Pacific Ocean for those that are native to that region. It would be further preferable if the additive was derived from macroalgae that can be cultivated in the northwestern Atlantic Ocean for those that are native to that region. It would be further preferable if the macroalgae produced a high amount of biomass over a short growing period.

### SUMMARY

The present technology is a feedstock additive that improves ruminant performance with regard to one or more of growth rate, meat quality and meat quantity or milk quality. The feedstock additive also reduces methane production and reduces bacterial count in ruminants. The west coast feedstock additive is from at least one brown or red algae species native to the northeastern Pacific Ocean. The east coast feedstock additive is from at least one brown or red algae species native to the northwestern Atlantic Ocean. The additive is derived from macroalgae that can be cultivated in the northeastern Pacific Ocean for those that are native to that region. The additive is derived from macroalgae that can be cultivated in the northwestern Atlantic Ocean for those that are native to that region. Each macroalgae produces a high amount of biomass.

A method of reducing methanogenesis in a ruminant comprising drying a farmed macroalgae selected from the group consisting of Egregia menziesii, Neoagarum fimbriatum, Saccharina latissimi, Alaria marginate, Macrocystis pyrifera, Hedophyllum sessile, Callophyllis species, Opuntiella californica, Sarcodiotheca gaudichaudii, and Mazzaella splendins; adding the dried additive at between 1% and 5% dry weight to a feedstock to provide an enhanced feedstock; and feeding the enhanced feedstock to the ruminant.

The method may further comprise blanching the farmed macroalgae prior to drying.

In the method, the feedstock may comprise 50% barley straw:50% barley silage on a dry weight basis.

In the method, the dried additive may be fed at 5%.

In the method, the macroalgae may be selected from the group consisting of Egregia menziesii, Neoagarum fimbriatum, Alaria marginate, and Hedophyllum sessile.

In the method, the macroalgae may be selected from the group consisting of Callophyllis species, Opuntiella californica, Sarcodiotheca gaudichaudii, and Mazzaella splendins.

In another embodiment, a method of preparing a feed additive is provided for ruminants, the method comprising growing at least one macroalgae species in an ocean-based farm; harvesting the macroalgae in the fall or the spring; and drying the macroalgae to produce the feed additive.

The method may further comprise blanching the macroalgae prior to drying.

The method may further comprise freezing the macroalgae prior to drying.

The method may further comprise powdering the macroalgae after drying.

In the method, the macroalgae may be harvested in the fall.

In the method, the macroalgae may be selected from the group consisting of Egregia menziesii, Neoagarum fimbriatum, Saccharina latissimi, Alaria marginate, Macrocystis pyrifera, Hedophyllum sessile, Callophyllis species, Opuntiella californica, Sarcodiotheca gaudichaudii, and Mazzaella splendins.

In the method, the macroalgae may be selected from the group consisting of Egregia menziesii, Neoagarum fimbriatum, Alaria marginate, and Hedophyllum sessile.

In the method, at least two macroalgae may be mixed to provide the feed additive.

In the method, the macroalgae may be selected from the group consisting of Callophyllis species, Opuntiella californica, Sarcodiotheca gaudichaudii, and Mazzaella splendins.

In the method, at least two macroalgae may be mixed to provide the feed additive.

In another embodiment, a feed additive is provided for adding to a ruminant feedstock, the feed additive comprising a dried, powdered mixture of at least two macroalgae selected from the group consisting of farm-grown Egregia menziesii, Neoagarum fimbriatum, Saccharina latissimi, Alaria marginate, Macrocystis pyrifera, Hedophyllum sessile, Callophyllis species, Opuntiella californica, Sarcodiotheca gaudichaudii, and Mazzaella splendins.

In the feed additive the macroalgae may be blanched to provide a blanched, dried and powdered mixture.

In the feed additive the macroalgae may be Saccharina latissimi and Alaria marginate.

In the feed additive the cultivated macroalgae may be cultivated in an ocean-based farm.

In another embodiment, a method of reducing Escherichia coli in a ruminant is provided, the method comprising drying a farmed macroalgae selected from the group consisting of Egregia menziesii, Neoagarum fimbriatum, Saccharina latissimi, Alaria marginate, Macrocystis pyrifera, Hedophyllum sessile, Callophyllis species, Opuntiella californica, Sarcodiotheca gaudichaudii, and Mazzaella splendins; adding the dried additive at between 1% and 5% dry weight to a feedstock to provide an enhanced feedstock; and feeding the enhanced feedstock to the ruminant.

The method may further comprise blanching the farmed macroalgae prior to drying.

In the method, the feedstock may comprise 50% barley straw:50% barley silage on a dry weight basis.

In the method, the dried additive may be fed at 5%.

In the method, the farmed macroalgae may be selected from the group consisting of Egregia menziesii, Neoagarum fimbriatum, Alaria marginate, and Hedophyllum sessile.

In the method, the farmed macroalgae may be selected from the group consisting of Callophyllis species, Opuntiella californica, Sarcodiotheca gaudichaudii, and Mazzaella splendins.

In yet another embodiment, a method of reducing Escherichia coli in a ruminant is provided, the method comprising: drying a farmed macroalgae selected from the group consisting of Chondrus crispus, Laminaria longicruris, Ascophyllum nodosum, Laminaria digitata, Fucus serratus, Fucus vesiculosus, Alaria esculenta, and Saccharina latissimi; adding the dried additive at between 1% and 5% dry weight to a feedstock to provide an enhanced feedstock; and feeding the enhanced feedstock to the ruminant.

The method may further comprise blanching the farmed macroalgae prior to drying.

In the method, the feedstock may comprise 50% barley straw:50% barley silage on a dry weight basis.

In the method, the dried additive may be fed at 5%.

In yet another embodiment, a feed additive for adding to a ruminant feedstock is provided, the feed additive comprising a dried, powdered mixture of at least two macroalgae selected from the group consisting of cultivated Chondrus crispus, Laminaria longicruris, Ascophyllum nodosum, Laminaria digitata, Fucus serratus, Fucus vesiculosus, Alaria esculenta, and Saccharina latissimi.

In the feed additive, the macroalgae may be blanched to provide a blanched, dried and powdered mixture.

In the feed additive, the macroalgae may be cultivated macroalgae.

In the feed additive, the cultivated macroalgae may be cultivated in an ocean-based farm.

In yet another embodiment, a method of improving growth and quality performance in a ruminant is provided, the method comprising: drying a farmed macroalgae selected from the group consisting of Saccharina latissimi, Alaria marginate, Macrocystis pyrifera; adding the dried additive at between 1% and 5% dry weight to a feedstock to provide an enhanced feedstock; and feeding the enhanced feedstock to the ruminant.

The method may further comprise blanching the farmed macroalgae prior to drying.

In the method, the feedstock may comprise 50% barley straw:50% barley silage on a dry weight basis.

In the method, the dried additive may be fed at 5%.

In another embodiment, a method of improving growth and quality performance in a ruminant is provided, the method comprising: drying a farmed macroalgae selected from the group consisting of drying a farmed macroalgae selected from the group consisting of Ascophyllum nodosum, Alaria esculenta, and Saccharina latissimi; adding the dried additive at between 1% and 5% dry weight to a feedstock to provide an enhanced feedstock; and feeding the enhanced feedstock to the ruminant.

The method may further comprise blanching the farmed macroalgae prior to drying.

In the method, the feedstock may comprise 50% barley straw:50% barley silage on a dry weight basis.

In the method, the dried additive may be fed at 5%.

### DESCRIPTION

Except as otherwise expressly provided, the following rules of interpretation apply to this specification (written description and claims): (a) all words used herein shall be construed to be of such gender or number (singular or plural) as the circumstances require; (b) the singular terms “a”, “an”, and “the”, as used in the specification and the appended claims include plural references unless the context clearly dictates otherwise; (c) the antecedent term “about” applied to a recited range or value denotes an approximation within the deviation in the range or value known or expected in the art from the measurements method; (d) the words “herein”, “hereby”, “hereof”, “hereto”, “hereinbefore”, and “hereinafter”, and words of similar import, refer to this specification in its entirety and not to any particular paragraph, claim or other subdivision, unless otherwise specified; (e) descriptive headings are for convenience only and shall not control or affect the meaning or construction of any part of the specification; and (f) “or” and “any” are not exclusive and “include” and “including” are not limiting. Further, the terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. Where a specific range of values is provided, it is understood that each intervening value, to the tenth of the unit of the lower limit unless the context clearly dictates otherwise, between the upper and lower limit of that range and any other stated or intervening value in that stated range, is included therein. All smaller sub ranges are also included. The upper and lower limits of these smaller ranges are also included therein, subject to any specifically excluded limit in the stated range.

Unless defined otherwise, all technical and scientific terms used herein have the same meaning as commonly understood by one of ordinary skill in the relevant art. Although any methods and materials similar or equivalent to those described herein can also be used, the acceptable methods and materials are now described.

The seaweeds in one part of this work are all native to the northeastern Pacific Ocean (California coast up to and including the Alaska coast) and in one embodiment are and in another embodiment are cultivated in farms in the Pacific Northwest. The farms are preferably ocean-based. The seaweeds belonging to the class Florideophyceae (red algae) are as follows: Callophyllis spp., which is native to the central coast of British Columbia; Opuntiella californica, which is native to the central coast of British Columbia; Sarcodiotheca gaudichaudii, which is native to the central coast of British Columbia; and Mazzaella splendins, which is found from Alaska to Baja California. Other species include Palmaria mollis, hecantensis and callophylloides.

The seaweeds belonging to the class Phaeophycaea (brown algae) are as follows: Egregia menziesii (feather boa kelp), which is native to the coastline of western North America from Alaska to Baja California; Neoagarum fimbriatum (fringed sieve kelp), which is native to the northeast coast of the Pacific; Saccharina latissima (sugar kelp), which is found on both sides of the Pacific and is native to the British Columbia coastline; Alaria marginata (winged kelp), which is found from Alaska to California and is native to the British Columbia coastline; Macrocystis pyrifera (giant kelp), which is found from Baja California north to southeast Alaska, and is also found in the southern oceans near South America, South Africa, Australia, and New Zealand; and Hedophyllum sessile (sea cabbage [kelp]) which is native to the northeast Pacific coast (native to the British Columbia coastline). These are all large macroalgae and produce a high amount of biomass over a short growing period. All are cultivated in ocean-based seaweed farms on the northwest coast of the Pacific Ocean.

Other species include Ulva californica, lactuca, lobata, rigida, stenophylla and taeniata.

The seaweeds in the other part of this work are all native to northwestern Atlantic Ocean (the Virginia coast to the east coast of Nunavut) and in one embodiment are collected and in another embodiment are cultivated in farms in the northwestern Atlantic Ocean. The farms are preferably ocean-based.

The seaweed belonging to the class Florideophyceae (red algae) are as follows: Chondrus crispus (Irish moss), which is native to the North Atlantic; and Palmaria palmata (true Dulse). Porphyra umbilicalis is also a redalgae that is found in the northwest Atlantic but is in the Bangiophyceae class.

The seaweeds belonging to the class Phaeophycaea (brown algae) and are native to the Northwest Atlantic are as follows: Laminaria longicruris; Laminaria digitata; Ascophyllum nodosum; Saccharina longicruris; Saccharina nigripes; Fucus serratus; Fucus vesiculosus; Laminaria digitata; Agarum cribrosum; Alaria esculenta; and Saccharina latissimi.

The use of macroalgae that are cultivated in farms that are located in the ocean provides a number of advantages. Contamination is reduced both by monitoring for biofouling as well as during the harvesting process and post-processing. During the harvesting process, harvesters are instructed to cut the seaweeds to remove as much biofouling as possible. In many cases entire blades are harvested so there is not a lot of contamination.

Using local species will reduce the potential introduction of invasive or non-endemic species. Local species, particularly kelps, produce a very high biomass which is further increased through cultivation as the macroalgae are retained on lines slightly below the surface of the water where there is good light quality and quantity. Further, for intertidal species, which spend a part of their time out of water, being cultivated in the water increases biomass over the biomass increase for wild macroalgae.

### Performance—Phase One

Two separate feeding trials were performed following the same general methodology. S. latissima, A. marginata, and M. pyrifera were provided as blanched and unblanched feedstock additive. A maximum of 5% additive (dry weight) inclusion was studied over two 90 day feeding periods, representing 500 g dried seaweed/animal/day. This required 900 kg dried seaweed material for 20 animals, which required 10 tonnes of harvested biomass per species. Prior to feeding trial start date, 20 cattle per species of seaweed were identified as 16 weeks away from harvest (1100-1400 pounds live weight). This group was split into two treatments: control (n=10) and seaweed additive (n=10). Each cohort was isolated in separate pens equipped with sleeping stalls, equipment accesses for cleaning, two GrowSafe® feeders and one GrowSafe waterer, and GreenFeed® automated emissions detector access. Each trial took 120 days. The week 1 was used to adapt the animal subjects to the pen, and weeks 2-4 was used to train the animal on the GreenFeed. Starting in week 5, the blanched seaweed additive was introduced into the feed (Experiment 1) or the unblanched seaweed additive was introduced into the feed (Experiment 2). For Experiment 1, the seaweed additive was mixed directly with the food at 5% using a mixing wagon and loaded into the GrowSafe feed bunks of the additive group. For Experiment 2, the seaweed additive was mixed directly with the food at 5% using a mixing wagon and loaded into the GrowSafe feed bunks of the additive group. Gas measurements were collected in alternating weeks using the GreenFeed systems for each group during the study. This strategy enabled us to obtain methane measurements for each group (control and additive) over 16 weeks total of emissions data over the course of each experiment.

The GrowSafe system identifies each animal by their Radio Frequency Identification (RFID) tag and uses automated weight measurements to track individual feed intake and weight gain over specified time periods. The weight data collected is automatically aggregated, stored wirelessly, and processed via specific algorithms to produce trial reports that include average daily gain (ADG), dry matter intake (DMI) and residual feed intake (RFI), among other useful indicators of performance.

Carcass metrics will be collected at the slaughterhouse. Rib eye steaks (longissimus dorsi) will be collected from each animal after slaughter and aging of the meat for the appropriate length of time. The complete fatty acid profile will be determined by conventional fatty acid methyl ester analysis by Gas Chromatography-Flame Ionization Detector by the Lipid Analytical Laboratory (Guelph, Ontario). Briefly, fats present in the beef samples will be hydrolyzed and the resulting free fatty acids methylated using a two-step saponification/methylation procedure. Although saturated fatty acids and monounsaturated fatty acids represent the majority of fat in beef, a detailed fatty acid profile provides additional info about the health quality of the meat for the consumer. For example, the consumption of n-6 polyunsaturated fatty acids and trans fatty acids should be limited while n-3 polyunsaturated fatty acids intake should be increased.

### Performance—Phase 2

Two separate feeding trials were performed following the same general methodology. S. latissima, A. marginata, and M. pyrifera were provided as blanched and unblanched feedstock additive. The additive was included at 10% additive (dry weight). Prior to feeding trial start date, 20 cattle per species of seaweed were identified as 16 weeks away from harvest (1100-1400 pounds live weight). This group was split into two treatments: control (n=10) and seaweed additive (n=10). Each cohort was isolated in separate pens equipped with sleeping stalls, equipment accesses for cleaning, two GrowSafe® feeders and one GrowSafe waterer, and GreenFeed® automated emissions detector access. Each trial took 120 days. The week 1 was used to adapt the animal subjects to the pen, and weeks 2-4 was used to train the animal on the GreenFeed. Starting in week 5, the blanched seaweed additive was introduced into the feed (Experiment 1) or the unblanched seaweed additive was introduced into the feed (Experiment 2). For Experiment 1, the seaweed additive was mixed directly with the food at 5% using a mixing wagon and loaded into the GrowSafe feed bunks of the additive group. For Experiment 2, the seaweed additive was mixed directly with the food at 5% using a mixing wagon and loaded into the GrowSafe feed bunks of the additive group. Gas measurements were collected in alternating weeks using the GreenFeed systems for each group during the study. This strategy enabled us to obtain methane measurements for each group (control and additive) over 16 weeks total of emissions data over the course of each experiment.

The GrowSafe system identifies each animal by their Radio Frequency Identification (RFID) tag and uses automated weight measurements to track individual feed intake and weight gain over specified time periods. The weight data collected is automatically aggregated, stored wirelessly, and processed via specific algorithms to produce trial reports that include average daily gain (ADG), dry matter intake (DMI) and residual feed intake (RFI), among other useful indicators of performance.

Carcass metrics will be collected at the slaughterhouse. Rib eye steaks (longissimus dorsi) will be collected from each animal after slaughter and aging of the meat for the appropriate length of time. The complete fatty acid profile will be determined by conventional fatty acid methyl ester analysis by Gas Chromatography-Flame Ionization Detector by the Lipid Analytical Laboratory (Guelph, Ontario). Briefly, fats present in the beef samples will be hydrolyzed and the resulting free fatty acids methylated using a two-step saponification/methylation procedure. Although saturated fatty acids and monounsaturated fatty acids represent the majority of fat in beef, a detailed fatty acid profile provides additional info about the health quality of the meat for the consumer. For example, the consumption of n-6 polyunsaturated fatty acids and trans fatty acids should be limited while n-3 polyunsaturated fatty acids intake should be increased.

### Performance—Phase 3

Two separate feeding trials were performed following the same general methodology. Ascophyllum nodosum, Alaria esculenta, and Saccharina latissimi cultivated in the northwest Atlantic were provided as blanched and unblanched feedstock additive. The additive was included at 10% additive (dry weight). Prior to feeding trial start date, 20 cattle per species of seaweed were identified as 16 weeks away from harvest (1100-1400 pounds live weight). This group was split into two treatments: control (n=10) and seaweed additive (n=10). Each cohort was isolated in separate pens equipped with sleeping stalls, equipment accesses for cleaning, two GrowSafe® feeders and one GrowSafe waterer, and GreenFeed® automated emissions detector access. Each trial took 120 days. The week 1 was used to adapt the animal subjects to the pen, and weeks 2-4 was used to train the animal on the GreenFeed. Starting in week 5, the blanched seaweed additive was introduced into the feed (Experiment 1) or the unblanched seaweed additive was introduced into the feed (Experiment 2). For Experiment 1, the seaweed additive was mixed directly with the food at 5% using a mixing wagon and loaded into the GrowSafe feed bunks of the additive group. For Experiment 2, the seaweed additive was mixed directly with the food at 5% using a mixing wagon and loaded into the GrowSafe feed bunks of the additive group. Gas measurements were collected in alternating weeks using the GreenFeed systems for each group during the study. This strategy enabled us to obtain methane measurements for each group (control and additive) over 16 weeks total of emissions data over the course of each experiment.

The GrowSafe system identifies each animal by their Radio Frequency Identification (RFID) tag and uses automated weight measurements to track individual feed intake and weight gain over specified time periods. The weight data collected is automatically aggregated, stored wirelessly, and processed via specific algorithms to produce trial reports that include average daily gain (ADG), dry matter intake (DMI) and residual feed intake (RFI), among other useful indicators of performance.

Carcass metrics will be collected at the slaughterhouse. Rib eye steaks (longissimus dorsi) will be collected from each animal after slaughter and aging of the meat for the appropriate length of time. The complete fatty acid profile will be determined by conventional fatty acid methyl ester analysis by Gas Chromatography-Flame Ionization Detector by the Lipid Analytical Laboratory (Guelph, Ontario). Briefly, fats present in the beef samples will be hydrolyzed and the resulting free fatty acids methylated using a two-step saponification/methylation procedure. Although saturated fatty acids and monounsaturated fatty acids represent the majority of fat in beef, a detailed fatty acid profile provides additional info about the health quality of the meat for the consumer. For example, the consumption of n-6 polyunsaturated fatty acids and trans fatty acids should be limited while n-3 polyunsaturated fatty acids intake should be increased.

### Example 1

Following inclusion of the dry seaweed to the feedstock at 5% weight to weight, it is expected that inclusion of seaweed in the cattle diet will lead to an increase an average daily gain and improved meat quality including a change in fatty acid profiles, more specifically, a decrease in the n-6 polyunsaturated fatty acids and an increase in the n-3 polyunsaturated fatty acids. For dairy cattle, the fatty acid profiles of the milk is also expected to change, again with a decrease in the n-6 polyunsaturated fatty acids and an increase in the n-3 polyunsaturated fatty acids.

### Example 2

Following inclusion of the dry seaweed to the feedstock at 10% weight to weight, it is expected that inclusion of blanched seaweed in the cattle diet will lead to an increase an average daily gain and improved meat quality including a change in fatty acid profiles, more specifically, a decrease in the n-6 polyunsaturated fatty acids and an increase in the n-3 polyunsaturated fatty acids. For dairy cattle, the fatty acid profiles of the milk is also expected to change, again with a decrease in the n-6 polyunsaturated fatty acids and an increase in the n-3 polyunsaturated fatty acids. The unblanched seaweed is expected to either have no effect or have a deleterious effect.

### Immuno-Stimulation and Reduction in Bacterial Count

E. coli O157:H7 causes significant losses during the finishing stages of meat production. Two separate feeding trials were performed following the same general methodology to determine the effect of a feedstock additive on E. coli. O157:H7. The feed additive was prepared from one or more of: Egregia menziesii; Neoagarum fimbriatum; Saccharina latissima; Alaria marginata; Macrocystis pyrifera; Hedophyllum sessile; Callophyllis spp.; Opuntiella californica; Sarcodiotheca gaudichaudii; and Mazzaella splendins for the species cultivated in the northeast Pacific. For species cultivated in the northwest Atlantic, the macroalgae source follows: Chondrus crispus; Laminaria longicruris; Ascophyllum nodosum; Laminaria digitata; Fucus serratus; Fucus vesiculosus; Alaria esculenta; and Saccharina latissimi. They were provided as blanched feedstock additives. A maximum of 5% additive (dry weight) inclusion was studied over two 90 day feeding periods, representing 500 g dried seaweed/animal/day/seaweed species. Prior to feeding trial start date, 20 cattle per seaweed species were identified as 16 weeks away from harvest (1100-1400 pounds live weight). This group was split into four treatments: control without E. coli O157:H7 inoculation (n=5); control with E. coli O157:H7 inoculation (n=5); seaweed additive without E. coli O157:H7 inoculation (n=5); and seaweed additive with E. coli O157:H7 inoculation (n=5). The treatments commenced 7 days after E. coli O157:H7 inoculation and fecal shedding patterns were examined over 14 weeks. Water, water-trough interface, feed and fecal pat samples were also collected weekly and cultured for E. coli O157:H7.

### Example 3

The results are expected to show a reduction in E. coli O157:H7 in the cattle receiving the feedstock additive.

### Methane Reduction

The rumen simulation technique (RUSITEC) was used to mimic in vivo digestion in vitro. Rumen fluid was obtained from three cannulated beef heifers fed the same diet as the control substrate for each study. The rumen fluid was collected and processed according to Saleem et al. The incubation was initiated by prefilling each fermenter with 180 mL of pre-warmed McDougall's buffer and 720 ml of strained rumen fluid. One nylon bag containing 20 grams (g) of mixed solid rumen digesta, and one nylon bag containing 10 g dry matter of the experimental diet was allocated to each fermenter. After 24 h incubation, the nylon bag containing rumen digesta was replaced by a nylon bag containing another bag of allocated diet and thereafter, one bag was replaced daily such that each bag is incubated for 48 h. The experimental period was 15 days with d 1-7 used for adaptation and day 8-15 used for measurements. The disappearance of dry matter (DM), organic matter (OM), neutral detergent fibre (NDF), and crude protein (CP) was assessed over the 48 hour incubation period. After 48 hour fermentation, feed bags was removed, washed in cold running water, and then dried at 55° C. for 48 hours for determination of DMD. After drying, residues were ground through a 1 millimeter screen and analyzed for organic matter (OM), neutral detergent fibre (NDF), and crude protein (CP).

Gas volume and concentration of carbon dioxide, hydrogen and methane, as well as fermenter volatile fatty acid (VFA) concentration, pH and ammonia was determined every 24 hours. Concentrations of CH4, O₂, H₂ and CO₂ and VFA was determined using a gas chromatography, and ammonia concentration was determined using the phenol-hypochlorite method.

The in vitro method allowed for rapid assessment of parameters including percent additive (1%, 2%, 4% and 5%) and macroalgae source: Egregia menziesii; Neoagarum fimbriatum; Saccharina latissima; Alaria marginata; Macrocystis pyrifera; Hedophyllum sessile; Callophyllis spp.; Opuntiella californica; Sarcodiotheca gaudichaudii; and Mazzaella splendins for the species cultivated in the northeast Pacific. For species cultivated in the northwest Atlantic, the macroalgae source follows: Chondrus crispus; Laminaria longicruris; Ascophyllum nodosum; Laminaria digitata; Fucus species; Alaria esculenta; and Saccharina latissimi. Other parameters were harvest date (fall versus spring), blanched or unblanched, and base feedstock, which was forage (50% barley straw:50% barley silage, dry matter basis) or grain (50% barley:50% corn, dry matter basis).

### Example 4

It is expected that the fall harvest will produce superior results, and that blanching is superior to not blanching. The preferred base feedstock is expected to be a forage feedstock and the preferred percentage of additive will be 5%. It is also expected that not all of the macroalgae will lead to a reduction in methanogensis.

While example embodiments have been described in connection with what is presently considered to be an example of a possible most practical and/or suitable embodiment, it is to be understood that the descriptions are not to be limited to the disclosed embodiments, but on the contrary, is intended to cover various modifications and equivalent arrangements included within the spirit and scope of the example embodiment. Those skilled in the art will recognize or be able to ascertain using no more than routine experimentation, many equivalents to the specific example embodiments specifically described herein. Such equivalents are intended to be encompassed in the scope of the claims, if appended hereto or subsequently filed.

1. A method of reducing methanogenesis in a ruminant, the method comprising drying a macroalgae native to the northeastern Pacific Ocean selected from the group consisting of Egregia menziesii, Neoagarum fimbriatum, Saccharina latissimi, Alaria marginate, Hedophyllum sessile, Callophyllis species, Opuntiella californica, Sarcodiotheca gaudichaudii, and Mazzaella splendins, to provide a dried additive; adding the dried additive at between 1% and 5% dry weight to a feedstock to provide an enhanced feedstock; and feeding the enhanced feedstock to the ruminant.

2. The method of claim 1, further comprising blanching the macroalgae prior to drying.

3. The method of claim 2, wherein the feedstock comprises 50% barley straw:50% barley silage on a dry weight basis.

4. The method of claim 3, wherein the dried additive is fed at 5%.

5. The method of claim 4, wherein the macroalgae is selected from the group consisting of Egregia menziesii, Neoagarum fimbriatum, Alaria marginate, and Hedophyllum sessile.

6. The method of claim 4, wherein the macroalgae is selected from the group consisting of Callophyllis species, Opuntiella californica, Sarcodiotheca gaudichaudii, and Mazzaella splendins.

7. A method of preparing a feed additive, the method comprising cultivating, in a farm in an ocean, at least one macroalgae species, which is native to that ocean; harvesting the macroalgae in the fall or the spring; and drying the macroalgae to produce the feed additive.

8. The method of claim 7, further comprising blanching the macroalgae prior to drying.

9. The method of claim 7, wherein the macroalgae are suspended in the epipelagic zone of the ocean during cultivating.

10. (canceled)

11. (canceled)

12. The method of claim 9, wherein the macroalgae are selected from the group consisting of Egregia menziesii, Neoagarum fimbriatum, Saccharina latissimi, Alaria marginate, Hedophyllum sessile, Callophyllis species, Opuntiella californica, Sarcodiotheca gaudichaudii, and Mazzaella splendins.

13. The method of claim 12, wherein the macroalgae is selected from the group consisting of Egregia menziesii, Neoagarum fimbriatum, Alaria marginate, and Hedophyllum sessile.

14. The method of claim 13, wherein at least two macroalgae are mixed to provide the feed additive.

15. The method of claim 12, wherein the macroalgae is selected from the group consisting of Callophyllis species, Opuntiella californica, Sarcodiotheca gaudichaudii, and Mazzaella splendins.

16. The method of claim 15, wherein at least two macroalgae are mixed to provide the feed additive.

17. A feed additive for adding to a ruminant feedstock, the feed additive comprising a dried, powdered mixture of at least two macroalgae selected from the group consisting of cultivated Egregia menziesii, Neoagarum fimbriatum, Saccharina latissimi, Alaria marginate, Hedophyllum sessile, Callophyllis species, Opuntiella californica, Sarcodiotheca gaudichaudii, and Mazzaella splendins.

18. The feed additive of claim 17 wherein the macroalgae are blanched to provide a blanched, dried and powdered mixture.

19. The feed additive of claim 18 wherein the macroalgae are Saccharina latissimi and Alaria marginate.

20-43. (canceled)

44. The method of claim 1, further comprising cultivating the macroalgae in a farm in the northeastern Pacific Ocean and harvesting the macroalgae prior to drying.

45. The method of claim 44, wherein the macroalgae are suspended in an epipelagic zone of the northeastern Pacific Ocean during cultivating.
